(19) United States
(12) Patent Application Publication
(10) Pub. No.: US 2016/0317834 A1
(43) Pub. Date: Nov. 3, 2016

(54) LIGHT TREATMENT THERAPY FOR AN EYE

(71) Applicant: Polyphotonix Ltd, Sedgefield (GB)

(72) Inventors: Richard Anthony Kirk, Sedgefield (GB); Duncan John Hill, Sedgefield (GB); Martin Neil Holland, Sedgefield (GB)

(73) Assignee: Polyphotonix Ltd, Sedgefield (GB)

(21) Appl. No.: 15/143,317

(22) Filed: Apr. 29, 2016

(30) Foreign Application Priority Data
May 1, 2015 (GB) 1507581.5

(51) Int. Cl. A61N 5/06

(52) U.S. Cl. CPC A61N 5/0613; A61N 2005/0648; A61N 2005/0651; A61N 2005/0653; A61N 2005/0663

(57) ABSTRACT

A method of light treatment therapy of an eye. The method includes the step of administering light to the eye, the light having a wavelength of from 495 to 505 nm.

### CROSS REFERENCE TO RELATED APPLICATION

This application claims the benefit of and priority to United Kingdom Patent Application No. 1507581.5, filed May 1, 2015, which is incorporated herein by reference.

### FIELD

The present invention relates to a method of and mask for light treatment therapy of an eye. In particular, but not exclusively, the eye may be that of a healthy person.

Diabetes is a global epidemic and diabetic retinopathy (DR) is the most common complication of this condition. In DR progressive damage to the light sensitive tissue at the back of the eye, the retina, can lead to blindness.

Diabetic retinopathy is the result of microvascular retinal changes where hyperglycemia-induced intramural pericyte death and thickening of the basement membrane cause damage to the wall of blood vessels in the eye. This damage changes the formation of the blood-retinal barrier and also makes the retinal blood vessels become more permeable.

Small blood vessels, such as those in the eye, are particularly vulnerable to poor blood sugar control. An overaccumulation of glucose and/or fructose damages the blood vessels in the retina. Damaged blood vessels are likely to leak fluid and lipids onto the macula. This condition can therefore lead to impaired vision and ultimately blindness.

It would be beneficial to stop or at least slow the onset and development of diabetic retinopathy.

In accordance with a first aspect of the present invention there is provided a method of light treatment therapy of an eye, the method comprising the step of administering light to the eye, the light having a wavelength of from 470 to 585 nm.

The method of light treatment therapy of an eye may further be a treatment for one or more of a retinal disease, myopia, hyperopia, Age-Related Macular Degeneration, dry Age-Related Macular Degeneration, wet Age-Related Macular Degeneration, Diabetic Retinopathy, hypoxia, and hypoxia caused by dark adaptation.

The method of light treatment therapy of an eye may improve visual acuity. Visual acuity is typically the clearness and/or acuteness of vision. The eye may be an eye of a healthy person. The method may improve the eyesight of the healthy person.

The step of administering light to the eye may use a mask. The mask may be worn by a person when they are sleeping. The step of administering light to the eye may be through closed eye lids. The step of administering light to the eye may be done regularly for less than or equal to three calendar months and/or 90 days. There may be a recover period of less than or equal to one calendar month and/or 30 days when the light is not administered to the eye.

It may be an advantage of the present invention that the step of administering light to the eye may result in the selective activation of rods and/or rod cells in the eye.

The light may be generated by one or more of an electroluminescent emitter, light emitting device (LED), light emitting cell (LEC), light emitting electrochemical cell (LEEC), and organic light emitting device (OLED). The light is typically arranged to emit light towards the eye.

The mask typically covers at least one eye of a person wearing the mask. The mask normally covers both eyes of a person wearing the mask. It may be an advantage of the present invention that administering light to the eye may improve retinal activity of the at least one, typically both eyes and/or may improve visual acuity.

The step of administering light to the at least one eye, typically both eyes typically improves the oxygen environment around the at least one eye, typically both eyes. This may reduce the demand and/or requirement of the at least one eye, typically both eyes, for oxygen. The reduced demand may be demand of tissue of the at least one eye, typically both eyes, for oxygen. The reduced demand may allow the at least one eye, typically both eyes, to function more efficiently.

The at least one eye, typically both eyes, normally comprise a retina. The retina normally has a poor oxygen supply and a high oxygen demand. It may be an advantage of the present invention that the step of administering light to the at least one eye, typically both eyes may reduce the demand and/or requirement of the at least one eye, typically both eyes, for oxygen and thereby allow the eye to rest.

The metabolic rate of eye tissue of the at least one eye, typically both eyes, may remain at least substantially constant, typically constant. The step of administering light to the at least one eye, typically both eyes typically increases the oxygen concentration in eye tissue of the at least one eye, typically both eyes.

The light may have a wavelength of from 470 to 570 nm (nanometers) or from 495 to 505 nm. The wavelength of the light may peak at 504 nm or 498 nm. The light may be referred to photons.

The step of administering light to the eye having a wavelength of from 470 to 585 nm may last for from 0.5 to 12 hours, typically from 4 to 8 hours. The step of administering light to the eye having a wavelength of from 470 to 585 nm typically lasts for from 0.5 to 12 hours, typically from 4 to 8 hours when the person is asleep. The eye typically does not dark adapt when the person is asleep during the step of administering light to the eye.

The light may have a frequency of from 500 to 650 THz, typically from 526 to 606 THz. The light may have a photon energy of from 2.00 to 2.75 eV, typically from 2.17 to 2.50 eV. The light may have a luminance of from 30 to 100 cd/m², optionally from 30 to 80 cd/m², typically from 30 to 60 cd/m². The light may have a luminance of 50 cd/m² or 80 cd/m². The light may have a radiance from 0.06 to 0.2 mW/sr/m², optionally from 0.06 to 0.15 mW/sr/m².

The method of light treatment therapy of an eye may be referred to as a low-dose light therapy. The method of light treatment therapy may improve the psychomotor vigilance and/or psychological wellbeing of the treated person. The method of light treatment therapy may improve retinal structure and/or function of the eye of the person, may be a healthy person, being treated.

The method of light treatment therapy of an eye may have a positive, typically beneficial, effect on the treated person. The positive, typically beneficial, effect on the treated person may be measured by one or more outcome measures, including a psychomotor vigilance task (PVT), typically primary outcome, may be number of lapses (NL), may be response time (RT); Karolinska sleepiness scale; Pittsburgh sleep quality; depression index; best corrected visual acuity; contrast sensitivity; Cambridge Color Vision; multifocal electroretinogram; electrooculogram; microperimetry; and optical coherence tomography central subfield thickness.

In accordance with a second aspect of the present invention there is provided a method of improving eyesight in a healthy person, the method comprising the step of administering light to an eye, the light having a wavelength of from 470 to 585 nm.

The light may have a wavelength of from 470 to 570 nm (nanometers) or from 495 to 505 nm. The wavelength of the light may peak at from 498 to 504 nm. Typically the wavelength of the light may peak at 504 nm or 498 nm. The light may be referred to as photons.

The healthy person will typically not have one or more of a retinal disease, myopia, hyperopia, Age-Related Macular Degeneration, dry Age-Related Macular Degeneration, wet Age-Related Macular Degeneration, Diabetic Retinopathy, hypoxia, hypoxia caused by dark adaptation. The method of improving eyesight in the healthy person may improve visual acuity of the healthy person.

The optional features of the first, third and/or fourth aspects of the present invention can be incorporated into the second aspect of the present invention and vice versa.

In accordance with a third aspect of the present invention there is provided a method of preparing a healthy person for light treatment therapy of an eye, the method comprising the step of placing onto the person a mask having a light source for emitting light having a wavelength of from 470 to 585 nm.

The method may further include the step of selecting a healthy person who could benefit from the light treatment therapy. The healthy person may be referred to as a patient.

The light may have a wavelength of from 470 to 570 nm (nanometers) or from 495 to 505 nm. The wavelength of the light may peak at from 498 to 504 nm. Typically the wavelength of the light may peak at 504 nm or 498 nm. The light may be referred to as photons.

The optional features of the first, second and/or fourth aspects of the present invention can be incorporated into the third aspect of the present invention and vice versa.

In accordance with a fourth aspect of the present invention there is provided a mask for light treatment therapy of an eye, the mask comprising a light source for administering light to the eye, the light having a wavelength of from 470 to 585 nm.

The optional features of the first, second and/or third aspects of the present invention can be incorporated into the fourth aspect of the present invention and vice versa.

There is also herein described a method of operating a medical apparatus for emitting radiation towards an area to be treated of a patient, the method comprising the steps of:
determining a radiation treatment program for a patient;
inputting instructions indicative of the treatment program into the medical apparatus; and
controlling a radiation source to emit electromagnetic radiation according to the instructions.

The mask according to the fourth aspect of the present invention may be the apparatus for emitting radiation towards an area to be treated of a patient.

There is also herein described a method of assembling an apparatus, the method comprising the steps of:
selecting one or more of a desired wavelength, intensity, waveform, and pulse modulation of radiation; and
setting a radiation source in accordance with one or more of the desired wavelength, intensity, waveform, and pulse modulation.

The method of assembling may further comprise the step of identifying the requirements of the user and selecting the desired wavelength in accordance with the identified requirements.

The mask according to the fourth aspect of the present invention may be the apparatus for emitting radiation towards an area to be treated of a patient.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

An embodiment of the invention will now be described by way of example only and with reference to the accompanying drawings, in which:

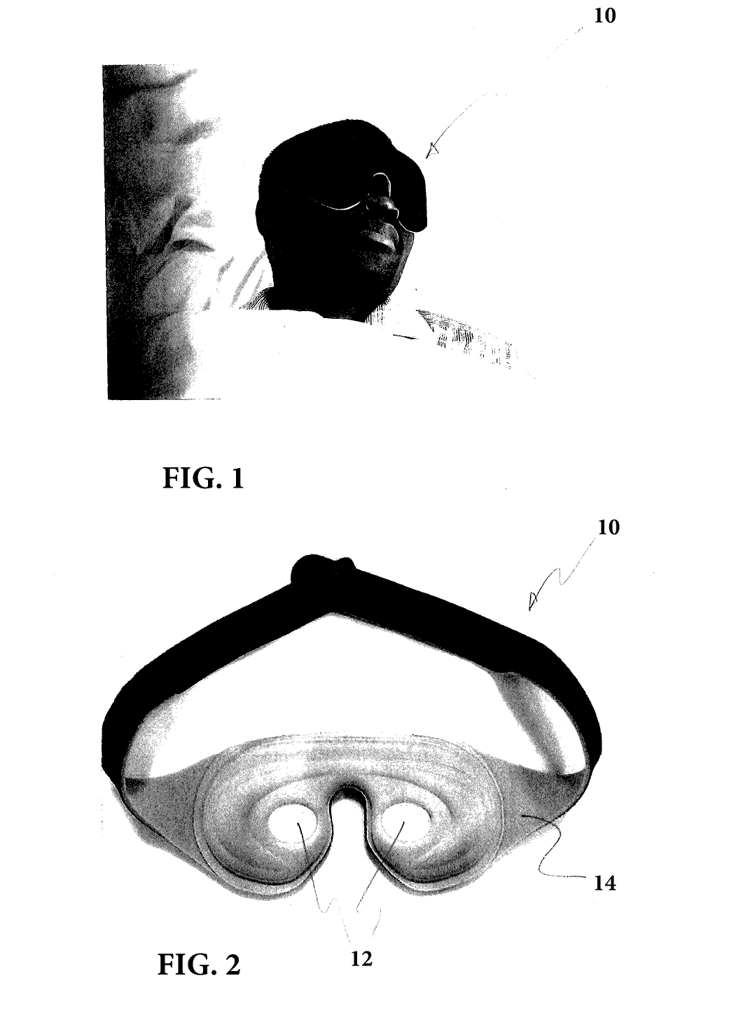

FIG. 1 is a perspective view of a mask in accordance with an aspect of the present invention; and

FIG. 2 is another perspective view of the mask.

### PARTICIPANTS AND METHODS

Participants were recruited into a single-centre prospective interventional clinical study in two groups: Group A 18-30 yrs and group B 50-70 yrs.

Masks emitting light with a luminance of 80 cd/m² and a peak at 504 nm developed for selective activation of rods up to a maximum of 8 hours during sleep were worn over closed lids each night for three months followed by a recovery period of one month.

Analysis of primary outcomes was corrected for multiple comparisons.

The study was conducted in accordance with Good Clinical Practice, the World Medical Association Declaration of Helsinki (“Ethical Principles for Medical Research Involving Human Subjects”). Approvals were by: the National Research Ethics Committee (13/WM/0011), the local Technique and Medical Devices Group and the Royal Liverpool and Broadgreen Hospitals NHS Trust (RLBUHT)(4407). Registration was with EUDRACT (2012-005188-29).

Study Design

The study was a single-centre, prospective, longitudinal non-commercial, interventional clinical study with a three month dosing period followed by a one month post dosing assessment.

Screening and Eligibility

Healthy adult volunteers with good general health from two age groups (group A: aged 18 to 30 years; group B: aged 50 to 70 years) were recruited at the Clinical Eye Research Centre, St Paul's Eye Unit, RLBUHT. All participants provided written informed consent. Key exclusion criteria were: any ocular or systemic disease that may affect the blood-retina barrier, unstable fixation on a microperimeter (fixation points <75% within 4°), self-reported history of sleep disturbances or disorders, PVT NL ≧17, self-reported history of depression or psychiatric disorders, use of psychoactive drugs (including the use of sleeping tablets).

Study Intervention

Sleep masks based on organic light emitting diode (OLED) technology were developed. FIGS. 1 and 2 show the mask 10 was provided in two parts, a plastic ‘Pod’ 12 and a soft cushioned fabric mask 14. The Pod contained the light sources which, when worn, emitted light into the eyes through closed lids. The size, position and shape of the Pod was chosen to cover the full range of movement for average eye positioning. The Pod operated on an internal clock allowing the mask to be activated for a maximum 8 hours between specified time points (typically a 14 hour window beginning at 8 pm). Touching a capacitive sensor on its side activated the Pod and switched on the light. The Pod was then slipped into the fabric mask and fastened over the face. The Pod contained sensors that detected when it was against the skin and recorded each minute of delivered dose.

The OLED spectrum with a peak of 504 nm was designed to closely match the scotopic response curve for selective activation of rods and delivers a light intensity of 2 Trolands at the retina to ensure suppression of dark adaptation and based on earlier work. Emission from the OLED was close to Lambertian and homogenous across the plane of the OLED, ensuring that movement of the eye during sleep did not result in substantial changes in illumination to the retina that could affect both treatment efficacy and disturb sleep. This allowed the brain to adapt to the presence of the light through Troxler's fading.

After training in mask usage eligible patients were provided with the OLED sleep mask, an instruction leaflet and a sleep diary to record sleep times and report their experience of the mask. Participants were instructed to wear the mask each night for three months. The mask was programmed assuming 8 hours maximum exposure each night for 30.5 days per month to provide each participant with up to 244 hours per month and 732 hours exposure over the duration of the study.

Participants attended monthly for four months. They were replaced if they withdrew before completing the first month visit. The mask and sleep diary were returned at each monthly visit up to the third month and a new mask and sleep diary provided for the following month. The hours of mask usage data accrued over the lifetime of the mask each month was extracted using contactless near field communication technology.

Study Procedures

The following assessments were performed at all visits.

Sleep Quality and General Psychological Wellbeing Test Series

A detailed assessment was conducted for fatigue-related changes in alertness associated with sleep loss, extended wakefulness, circadian misalignment, sleep quality and general psychological wellbeing. The psychomotor vigilance task (PVT) was used as a measure of loss of concentration and vigilance caused by sleep deprivation. Participants were asked to respond immediately with a button press when a coloured circle viewed on a computer screen briefly brightened at random intervals. The circle was displayed 130 times over an approximately 10 minute period. The dependent variables were the response time (msec) (PVT-RT) and the number of lapses (in which participants do not respond) (PVT-NL), both of which increased as an index of sleepiness. Based on normative data from healthy controls, we derived upper limits of normal for PVT: number of lapses mean+2 standard deviations (SD)≦16; response times mean+2 SD≦459, median+2 SD≦359.

The Karolinska Sleepiness Scale (KSS) questionnaire was used to test for changes in reported levels of sleepiness; rated on a scale ranging between 1 and 9 (mean 5.7±2.01), with higher scores indicating greater sleepiness. The Pittsburgh Sleep Quality Index (PSQI) a 19-item questionnaire assessed sleep quality on seven domains (sleep quality, sleep latency, sleep duration, habitual sleep efficiency, sleep disturbances, use of sleeping medications, daytime dysfunction) summed to provide a global score ranging between 0 and 21 (mean 7.4±5.1), higher scores indicating poorer sleep quality.

Psychomotor Vigilance Task Number of Lapses (PVT NL), Psychomotor Vigilance Task Response Time (PVT RT), the Karolinska Sleepiness Scale (KSS) and Pittsburgh Sleep Quality Index (PSQI) served as co-primary outcomes.

The Centre for Epidemiologic Studies Depression scale (CES-D), a self-reported instrument was used for assessing the symptoms of depression. Twenty items that consider mood, somatic complaints (including sleep quality), social interaction and motor functioning and responses are scored on a four-point Likert-scale. A standard cut-off score of 16 has been defined to assess depressive symptomatology (mean=10.24±9.67).

Ocular Function and Structure

Detailed medical and ocular history, standard ophthalmic examination, best-corrected visual acuity (BCVA) (Early Treatment Diabetic Retinopathy (ETDRS) letters read at 1 meter), contrast sensitivity (CS) on a Pelli Robson chart at 1 meter, fundus autofluorescence (AF), 19 segment multifocal electroretinogram (mfERG, Roland Retiscan), electrooculogram (EOG), microperimetry (MP, Nidek MP1), spectral domain optical coherence tomography (SD-OCT; Heidelberg Engineering GMbH) assessments were used.

Cambridge Colour vision Test ((CCT) Cambridge Research Systems) was performed to monitor colour vision changes quantitatively over time. Thresholds are measured in 10⁻⁴ u′v′ units. Normal range was defined as thresholds lower than 100×10⁻⁴ u′v′ units for the protan and deutan lines, and lower than 150×10⁻⁴ u′v′ units for the tritan line.

Adverse Events and Withdrawals

At each visit participants were specifically asked about adverse events. They were questioned on discomfort, any sleep disturbance, mood alterations or changes in wakefulness during the day. Reasons for withdrawal were sought. Adverse events were reviewed independently by two investigators and grouped into four categories.

Statistical Analysis

A formal sample size calculation was not possible due to lack of previously published data. Groups of 20 were selected based on accepted guidance for paired t tests.

STATA 13.1 was used to perform the statistical analysis. Linear regression was used to compare the changes from baseline to months three and four for all outcomes, each corrected for hours of mask wear. Where data were not normally distributed log-transformations were used. Means of changes were reported with 95% confidence limits.

A two-step method was used to adjust values of a for multiple comparisons for change from baseline to month three of the four co-primary variables. First, using a standard Bonferroni calculation, a level of significance of 0.05/4=0.013 was selected to ensure a family-wise error rate of 0.05. Then, to test the two hypotheses (groups A and B) for each co-primary outcome a Holm-Bonferroni procedure was used which compares the ordered p-values with the following corrected significance levels calculated for the study of 0.0063 and 0.013. P values are presented uncorrected and interpreted against the revised values of α.

Results

Of the 45 participants (21 in group A, 24 in group B), 11 (24%) withdrew, 5 (11%) before month one. Reasons for withdrawal given were sleep disturbances and mask intolerance. 30 of the remaining 40 (75%) who continued beyond month one reported ≧1 adverse event, mainly associated with mask discomfort and/or ocular symptoms. No serious adverse events occurred. Total mean mask wear in hours was lower in group A than in group B (26.67%, p<0.001).

Conclusions

The OLED sleep masks emitting light of 504 nm showed no significant safety signal apart from an impairment of vigilance tasks for which the effect size was small, well within the previously published variation and we believe not clinically significant. Sleep disturbance and light intolerance caused early withdrawal. Mask and ocular discomfort were commonly reported but tended to be tolerable and did not affect mask wear.

Retinal diseases such as myopia, diabetic retinopathy (DR), age-related macular degeneration (AMD) and retinal vein occlusion (RVO) are associated with changes in the retinal vasculature, leakage of blood vessels, macular edema and neovascularization. Most recent landmark treatment trials in these diseases have focused on modifying the VEGF pathway.

Results Tables

A total of 21 healthy volunteers were recruited in group A (18 to 30 years) and 24 in group B (50 to 70 years). Patient demographics and baseline values for study variables are presented in Table 1.

TABLE 1
Demographics and baseline values for 45 recruited participants

| | Group A | | | | Group B | | | |
|---|---|---|---|---|---|---|---|---|
| | n | mean (SD) | | Range | n | mean (SD) | | range |
| Age (years) | 21 | 22.52 | (3.66) | 18-30 | 24 | 56.96 | (5.07) | 50-68 |

| | | | | |
|---|---|---|---|---|
| Gender (m:f) | 21 | 12:9 | 24 | 7:17 |

| | | | | | | | | |
|---|---|---|---|---|---|---|---|---|
| PVT NL (number) | 21 | 4.55 | (4.57) | 0-18 | 24 | 4.29 | (5.21) | 0-24 |
| PVT RT (msec) | 21 | 340.52 | (46.78) | 276.15-456.63 | 24 | 340.52 | (46.78) | 276.15-456.63 |
| KSS (score) | 21 | 3.43 | (1.69) | 1-7 | 24 | 2.42 | (1.06) | 1-5 |
| PSQI (score) | 21 | 2.62 | (2.27) | 0-8 | 24 | 3.58 | (2.24) | 1-9 |
| CESD (score) | 21 | 4.33 | (3.77) | 0-16 | 24 | 3.71 | (4.28) | 0-13 |
| BCVA (letters) | 21 | 90.67 | (3.23) | 85-96 | 24 | 89.50 | (3.09) | 83-95 |
| CS (letters) | 20^{§} | 41.10 | (1.74) | 36-42 | 23^{§} | 40.87 | (1.91) | 35-42 |
| CCT protan | 21 | 57.43 | (18.73) | 22-95 | 23^{§} | 64.17 | (13.94) | 40-86 |
| CCT deutan | 21 | 51.38 | (17.60) | 26-97 | 23^{§} | 65.48 | (18.37) | 39-122 |
| CCT tritan | 21 | 64.43 | (20.66) | 33-106 | 23^{§} | 82.70 | (32.87) | 29-158 |
| mfERG amp 1 (μV) | 20^{§} | 66.40 | (16.75) | 36.10-108.00 | 24 | 66.16 | (12.96) | 44.90-99.50 |
| mfERG lat 1 (msec) | 20^{§} | 35.64 | (2.09) | 31.50-40.20 | 24 | 36.29 | (1.76) | 33.40-40.20 |
| EOG (Arden ratio) | 21 | 2.42 | (0.49) | 1.77-3.75 | 24 | 2.21 | (0.39) | 1.60-3.00 |
| MP1 | 18 | 19.51 | (1.08) | 15.60-20.00 | 23 | 19.32 | (1.25) | 15.40-20.00 |
| OCT CST (μm) | 21 | 277.33 | (19.52) | 238-319 | 24 | 282.71 | (19.63) | 254-319 |

^{§}one participant in each group was unable to perform some procedures
Legend:
BCVA = best corrected visual acuity;
CCT = Cambridge color vision test;
CESD = The Centre for Epidemiologic Studies Depression scale;
CS = contrast sensitivity;
EOG = electro-oculogram;
KSS = Karolinska Sleep Scale;
mfERG = multifocal electroretinogram;
MP1 = microperimetry;
OCT CST = optical coherence tomography central subfield thickness;
PSQI = Pittsburgh Sleep Quality Index;
PVT NL = Psychomotor Vigilance Task number of lapses; and
PVT RT = Psychomotor Vigilance Task response time.

Of the 45 participants who entered the study, 5 withdrew before month one and were replaced and 6 withdrew after completing month one study assessments. In both groups 17 participants completed all study visits and investigations. Reasons for withdrawal given at exit interview are given in Table 2. Light intolerance and sleep disturbance were cited by 1 participant in group A and 5 in group B. Other reasons given included an inability to perform study investigations for 1 participant and inability to continue attendance for 3. Data from the sleep diaries in those who withdrew recorded issues with the light in 3 participants.

TABLE 2
Reasons given for withdrawal of 11 participants by study visit

| | Reported reason for withdrawal | Group A | Group B |
|---|---|---|---|
| Before month 1 | intolerance of light and | 0 | 3 |
| | sleep disturbances | | |
| | unable to attend follow-up | 1 | 0 |
| | no reason given | 0 | 1 |
| After month 1 | intolerance of light and | 0 | 2 |
| | sleep disturbances | | |
| At month 2 | intolerance of light and | 1 | 0 |
| | sleep disturbances | | |
| | could not perform study | 0 | 1 |
| | investigations | | |
| | unable to attend follow-up | 2 | 0 |

Mean hours of sleep mask wear, equivalent to exposure to light therapy, are shown in Table 3. The time the mask was worn from baseline to month three was lower at 410±125 hours in group A compared to 559±87.4 hours in group B (p<0.001). In group A the amount of mask wear was more variable and stayed stable while in group B mask wear improved and became more consistent (Table 3). On average younger participants only received 56% compared to older participants who received 76% of the stipulated light-dose over the duration of the study.

TABLE 3
Hours of sleep mask wear (and exposure to light therapy) by month and
group

| | Group A | | | Group B | | |
|---|---|---|---|---|---|---|
| Time from baseline | n | mean (SD) | range | n | mean (SD) | range |
| Month 1 | 19 | 143 (52.6) | 22.3-199.6 | 20 | 174.2 (48.4) | 74.4-246.1 |
| Month 2 | 19 | 122.7 (55.0) | 33.9-195.0 | 18 | 185.2 (37.0) | 107.8-233.7 |
| Month 3 | 17 | 131.7 (55.0) | 21.6-215.7 | 17 | 188.9 (30.3) | 129.4-233.1 |
| Total* | 17 | 410.0 (125.1) | 119.7-570 | 17 | 559.1 (87.4) | 394.1-680.0 |

*Total mask hours calculated only in those who completed all three months.

Tables 4 and 5 show the changes in study variables at months three and four respectively compared to baseline for 17 participants in each group who completed three months of sleep mask wear. An effect of hours of mask wear was also explored. For the primary variables at month three, the PVT RT deteriorated in both age groups: group A 24.39 (7.25%), group B 25.39 (7.65%) and this was statistically significant at p<0.001 for Group A. PVT NL also deteriorated significantly in Group A (1.96 (43.27%), p=0.005). RT stayed depressed at month four while NL recovered.

Table 4 shows changes in sleep quality, psychological wellbeing, and ocular function and structure at month three in 36 participants who completed three months of sleep mask wear. Data are presented as means with standard deviation (SD) or 95% confidence intervals and uncorrected p values for overall change from baseline and per 100 hours of sleep mask wear.

Table 5 shows changes in sleep quality, psychological wellbeing, and ocular function and structure one month after discontinuing sleep mask wear in 36 participants who completed three months of wear. Data are presented as means with standard deviation (SD) of 95% confidence intervals and uncorrected p values for overall change from baseline and per 100 hours of sleep mask wear.

TABLE 4
Changes in sleep quality, psychological wellbeing and ocular function and structure at month three in 36 participants who
completed three months of sleep mask wear

| | Group A (n = 17) | | | Group B (n = 17) | | |
|---|---|---|---|---|---|---|
| | baseline | change at | change per 100 hours | baseline | change at month 3 | change per 100 hours |
| | (SD) | month 3 (95% CI) | (95% CI) | (SD) | (95% CI) | (95% CI) |
| | range | p value | p value | range | p value | p value |
| PVT NL | 4.53 | 1.96 | 0.28 | 4.30 | −0.17 | 0.13 |
| (number) | (4.69) | (0.63, 3.64) | (−0.63, 1.40) | (5.44) | (−1.15, 1.11) | (−1.24, 1.93) |
| | 0-18 | 0.005 | 0.55 | 0-24 | 0.75 | 0.88 |
| PVT RT(ms) | 336.41 | 24.39 | 3.38 | 332.10 | 25.39 | −3.30 |
| | (36.62) | (13.35, 39.12) | (−3.35, 13.73) | (50.5) | (6.71, 42.34) | (−22.45, 17.03) |
| | 270.89-418.43 | <0.001 | 0.35 | 276.1-456.6 | 0.02 | 0.67 |
| KSS (score) | 3.41 | 0.74 | 0.32 | 2.41 | 0.47 | 0.06 |
| | (1.80) | (−0.33, 1.86) | (−0.60, 1.24) | (1.12) | (−0.16, 1.10) | (−0.71, 0.84) |
| | 1-7 | 0.16 | 0.47 | 1-5 | 0.13 | 0.86 |
| PSQI (score) | 2.65 | 0.82 | 0.01 | 3.60 | 0.06 | −0.26 |
| | (2.32) | (−0.16, 1.81) | (−0.83, 0.85) | (2.23) | (−1.23, 1.35) | (−1.83, 1.32) |
| | 0-8 | 0.09 | 0.99 | 1-9 | 0.92 | 0.73 |
| CESD (score) | 4.30 | 1.35 | −0.95 | 4.20 | −0.88 | −1.07 |
| | (3.87) | (−0.52, 3.23) | (−2.47, 0.58) | (4.50) | (−3.13, 1.37) | (−3.76, 1.62) |
| | 0-16 | 0.15 | 0.21 | 0-13 | 0.42 | 0.49 |
| BCVA | 90.65 | 1.59 | 0.80 | 89.18 | 1.17 | 0.28 |
| (letters) | (3.32) | (−0.02, 3.19) | (−0.50, 2.10) | (3.28) | (−0.10, 2.45) | (−1.27, 1.83) |
| | 85-96 | 0.05 | 0.21 | 83-95 | 0.07 | 0.70 |
| CS (letters) | 41.29 | 0.35 | −0.15 | 41.18 | 0.17 | 0.31 |
| | (1.36) | (−0.37, 1.08) | (−0.76, 0.47) | (1.51) | (−0.64, 0.99) | (−0.67, 1.30) |
| | 37-42 | 0.32 | 0.63 | 36-42 | 0.65 | 0.66 |
| CCT protan | 60.29 | −3.41 | −2.46 | 61.56 | −7.75 | 6.77 |
| | (19.47) | (−12.35, 5.52) | (−9.99, 5.07) | (14.47) | (−19.93, 4.43) | (−7.25, 20.78) |
| | 22-95 | 0.43 | 0.49 | 40-86 | 0.19 | 0.32 |
| CCT deutan | 53.47 | 2.59 | −2.83 | 66.56 | −13.00 | 1.11 |
| | (18.57) | (−5.69, 10.87) | (−9.74, 4.09) | (19.19) | (−22.73, −3.27) | (−10.48, 12.71 |
| | 26-97 | 0.51 | 0.40 | 45-122 | 0.01 | 0.84 |
| CCT tritan | 64.12 | −2.47 | −2.76 | 78.06 | −4.06 | −5.04 |
| | (22.51) | (−16.02, 11.08) | (−14.25, 8.74) | (36.58) | (−28.84, 20.71) | (−34.46, 24.38) |
| | 33-106 | 0.70 | 0.62 | 29-158 | 0.73 | 0.71 |
| mfERG amp | 68.98 | 0.39 | −2.93 | 64.25 | 1.45 | 2.82 |
| 1 (μV) | (16.02) | (−8.43, 9.21) | (−10.31, 4.45) | (12.22) | (−3.99, 6.91) | (−3.68, 9.32) |
| | 41.5-108 | 0.92 | 0.41 | 44.9-88.6 | 0.58 | 0.37 |
| mfERG lat 1 | 35.81 | −1.18 | −0.84 | 36.25 | −0.29 | −0.21 |
| (msec) | (2.19) | (−2.14, −0.22) | (−1.52, −0.16) | (1.72) | (−1.05, 0.48) | (−1.14, 0.71) |
| | 31.5-40.2 | 0.02 | 0.02 | 33.4-39.2 | 0.44 | 0.63 |
| MP1 | 19.45 | 0.25 | 0.44 | 19.41 | −0.06 | 0.40 |
| | (1.13) | (−0.28, 0.78) | (0.08, 0.81) | (1.05) | (−0.73, 0.61) | (−0.38, 1.20) |
| | 15.6-20 | 0.33 | 0.02 | 16.6-20 | 0.86 | 0.30 |
| OCT CST | 274.53 | −0.53 | −1.63 | 286.53 | −2.82 | 0.12 |
| (μm) | (16.72) | (−2.60, 1.54) | (−3.16, −0.10) | (20.88) | (−5.62, −0.02) | (−3.31, 3.55) |
| | 238-308 | 0.60 | 0.038 | 255-319 | 0.05 | 0.94 |

Legend:
BCVA = best corrected visual acuity;
CCT = Cambridge color vision test;
CESD = The Centre for Epidemiologic Studies Depression scale;
CS = contrast sensitivity;
EOG = electro-oculogram;
KSS = Karolinska Sleep Scale;
mfERG = multifocal electroretinogram;
MP1 = microperimetry;
OCT CST = optical coherence tomography central subfield thickness;
PSQI = Pittsburgh Sleep Quality Index;
PVT NL = Psychomotor Vigilance Task number of lapses; and
PVT RT = Psychomotor Vigilance Task response time.

TABLE 5
Changes in sleep quality, psychological wellbeing, and ocular function and structure one month after discontinuing
sleep mask wear in 36 participants who completed three months of wear

| | Group A (n = 17) | | | Group B (n = 17) | | |
|---|---|---|---|---|---|---|
| | | | change per 100 | | | change per 100 |
| | baseline | change at month 3 | hours | baseline | change at month 3 | hours |
| | (SD) | (95% CI) | (95% CI) | (SD) | (95% CI) | (95% CI) |
| | range | p value | p value | range | p value | p value |
| PVT NL | 4.53 | 0.59 | 2.09 | 4.30 | 1.39 | −0.56 |
| (number) | (4.69) | (−0.26, 11.28) | (−0.78, 7.18) | (5.44) | (−0.95, 5.27) | (−2.39, 3.08) |
| | 0-18 | 0.07 | 0.41 | 0-24 | 0.27 | 0.88 |
| PVT RT | 336.41 | 28.02 | 10.25 | 332.10 | 27.66 | 0.00 |
| (ms) | (36.62) | (10.24, 46.70) | (−3.35, 24.39) | (50.5) | (10.11, 46.10) | (−22.45, 24.08) |
| | 270.89-418.43 | 0.005 | 0.45 | 276.1-456.6 | 0.01 | 0.66 |
| KSS | 3.41 | 0.41 | 0.44 | 2.41 | 0.29 | 0.05 |
| (score) | (1.80) | (−0.68, 1.50) | (−0.93, 0.94) | (1.12) | (−0.33, 0.92) | (−0.71, 0.81) |
| | 1-7 | 0.44 | 0.99 | 1-5 | 0.33 | 0.86 |
| PSQI (score) | 2.65 | 0.12 | 0.00 | 3.60 | 0.71 | −0.19 |
| | (2.32) | (−0.73, 0.97) | (−0.73, 0.73) | (2.23) | (−0.63, 2.04) | (−1.82, 1.44) |
| | 0-8 | 0.77 | 0.93 | 1-9 | 0.28 | 0.69 |
| CESD | 4.30 | 1.38 | −1.00 | 4.20 | 0.19 | −0.45 |
| (score) | (3.87) | (−1.05, 3.80) | (−2.95, 0.94) | (4.50) | (−2.65, 3.02) | (−3.81, 2.93) |
| | 0-16 | 0.24 | 0.29 | 0-13 | 0.88 | 0.78 |
| BCVA | 90.65 | 2.44 | 0.48 | 89.18 | 1.59 | −0.13 |
| (letters) | (3.32) | (0.68, 4.20) | (−1.04, 1.99) | (3.28) | (0.23, 2.95) | (−1.80, 1.53) |
| | 85-96 | 0.001 | 0.51 | 83-95 | 0.025 | 0.87 |
| CS | 41.29 | 0.56 | −0.33 | 41.18 | 0.06 | 0.43 |
| (letters) | (1.36) | (−0.26, 1.39) | (−1.03, 0.36) | (1.51) | (−0.47, 0.59) | (−0.17, 1.03) |
| | 37-42 | 0.17 | 0.32 | 36-42 | 0.82 | 0.15 |
| CCT protan | 60.29 | −8.88 | −2.05 | 61.56 | −2.19 | −8.34 |
| | (19.47) | (−9.78, 2.01) | (−11.31, 7.20) | (14.47) | (−14.14, 9.76) | (−5.10, 21.77) |
| | 22-95 | 0.10 | 0.64 | 40-86 | 0.70 | 0.21 |
| CCT deutan | 53.47 | −1.29 | 1.76 | 66.56 | −10.00 | 3.11 |
| | (18.57) | (−11.68, 9.10) | (−7.08, 10.60) | (19.19) | (−2.55, 2.55) | (−11.7717.98) |
| | 26-97 | 0.80 | 0.68 | 45-122 | 0.11 | 0.66 |
| CCT tritan | 64.12 | −3.47 | 3.86 | 78.06 | 0.19 | −0.66 |
| | (22.51) | (−22.11, 15.16) | (−11.94, 29.67) | (36.58) | (−18.07, 18.44) | (−22.52, 21.04) |
| | 33-106 | 0.70 | 0.61 | 29-158 | 0.98 | 0.94 |
| mfERG amp | 19.45 | 0.10 | 0.06 | 19.41 | 0.30 | 0.11 |
| 1 (μV) | (1.13) | (−0.28, 0.48) | (−0.26, 0.38) | (1.05) | (−0.14, 1.14) | (−0.81, 1.03) |
| | 15.6-20 | 0.59 | 0.69 | 16.6-20 | 0.30 | 0.79 |
| mfERG lat 1 | 68.98 | 1.70 | −2.93 | 64.25 | 7.81 | 2.82 |
| (msec) | (16.02) | (−7.35, 10.75) | (−10.31, 4.45) | (12.22) | (0.88, 14.74) | (−3.68, 9.32) |
| | 41.5-108 | 0.70 | 0.41 | 44.9-88.6 | 0.03 | 0.37 |
| MP1 | 35.81 | −1.22 | 1.56 | 36.25 | 0.08 | 3.10 |
| | (2.19) | (−2.14, −0.30) | (−6.14, 9.25) | (1.72) | (0.84, 1.01) | (−5.23, 11.42) |
| | 31.5-40.2 | 0.01 | 0.67 | 33.4-39.2 | 0.85 | 0.44 |
| OCT CST | 274.53 | 0.24 | −0.56 | 286.53 | 1.24 | 2.54 |
| (μm) | (16.72) | (−1.41, 1.88) | (−1.93, 0.81) | (20.88) | (−3.18, 5.65) | (−5.37, 5.44) |
| | 238-308 | 0.77 | 0.39 | 255-319 | 0.56 | 0.99 |

Legend:
BCVA = best corrected visual acuity;
CCT = Cambridge color vision test;
CESD = The Centre for Epidemiologic Studies Depression scale;
CS = contrast sensitivity;
EOG = electro-oculogram;
KSS = Karolinska Sleep Scale;
mfERG = multifocal electroretinogram;
MP1 = microperimetry;
OCT CST = optical coherence tomography central subfield thickness;
PSQI = Pittsburgh Sleep Quality Index;
PVT NL = Psychomotor Vigilance Task number of lapses; and
PVT RT = Psychomotor Vigilance Task response time.

Interpreting the secondary outcomes requires caution due to the multiple comparisons. In older participants there were reductions in scores for the three CCT values at month three with recovery at month four. These changes were consistent and statistically significant for the deutan channel (13.00 (19.5%) p=0.01).

For the other secondary variables there were no clinically important changes detected. There were small increases in BCVA which at month four were 2.44 letters in group A (p<0.001) and in Group B 1.59 letters (p=0.025). In the older participants there was a slight mean reduction in OCT CST (optical coherence tomography central subfield thickness) but at 2.82 μm this was not clinically significant.

The effect of duration of mask wear was tested against the primary and secondary variables. No clinically important effect was detected on variable scores associated with duration of recorded mask wear. There were small effects in the expected direction for mfERG latency, MP1 and OCT CST in Group A at month three.

Of the participants in each group, 75% who wore the sleep mask for at least one month reported at least one adverse event (Table 6). The majority of events were attributed to the fabric mask housing the OLED ‘Pod’. There were no serious adverse events.

TABLE 6
Adverse events reported by 40 participants who completed at
least one month of follow-up shown by participant and event

| | Group A | | Group B | |
|---|---|---|---|---|
| | participants | events | participants | events |
| Totals | 15 | 40 | 15 | 64 |
| Ocular | | | | |
| Red eyes | 0 | 0 | 2 | 3 |
| Eye soreness | 2 | 2 | 3 | 6 |
| Eye pain | 1 | 1 | 1 | 1 |
| Eyelid swelling | 1 | 1 | 3 | 4 |
| Pressure signs from mask | 0 | 0 | 1 | 1 |
| ‘Heavy eyelids’ | 0 | 0 | 1 | 1 |
| Itchy eyes | 1 | 1 | 2 | 2 |
| Dry eyes | 0 | 0 | 2 | 5 |
| Watery eyes | 1 | 1 | 1 | 1 |
| Loss of eyelashes | 0 | 0 | 1 | 1 |
| Double vision | 0 | 0 | 1 | 1 |
| Blurred vision | 1 | 1 | 0 | 0 |
| Photophobia | 1 | 1 | 0 | 0 |
| Corneal foreign body | 0 | 0 | 1 | 1 |
| Mask related adverse | | | | |
| events | | | | |
| General mask discomfort | 3 | 3 | 2 | 2 |
| Soreness around ears | 2 | 3 | 1 | 2 |
| Ear pain | 2 | 3 | 0 | 0 |
| Mask digging into bridge | 1 | 1 | 3 | 5 |
| of the nose | | | | |
| Mask pressing on eye | 1 | 1 | 1 | 1 |
| socket | | | | |
| Mask too hot on face | 4 | 6 | 1 | 3 |
| Due to light brightness | 2 | 3 | 1 | 1 |
| Rubbing of the mask | 1 | 2 | 2 | 2 |
| Velcro | | | | |
| Straps rubbing the ears | 1 | 2 | 1 | 1 |
| Label at the back of the | 0 | 0 | 1 | 1 |
| mask strap | | | | |
| Pressure on temporal | 1 | 1 | 0 | 0 |
| area | | | | |
| Pressure on eyes | 1 | 1 | | |
| Sleep disturbance | | | | |
| Change in sleep pattern | 0 | 0 | 1 | 1 |
| Due to mask slipping off | 1 | 1 | 1 | 1 |
| Other | | | | |
| Pneumonia | 0 | 0 | 1 | 1 |
| Viral respiratory | 0 | 0 | 1 | 1 |
| infection | | | | |
| Skin rash on the cheeks | 0 | 0 | 1 | 1 |
| (associated with viral | | | | |
| respiratory infection) | | | | |
| Gastroenteritis | 0 | 0 | 1 | 1 |
| Back pain | 0 | 0 | 1 | 1 |
| Migraine | 3 | 4 | 0 | 0 |
| Headaches | 1 | 1 | 2 | 2 |
| Dental pain (after tooth | 1 | 1 | 0 | 0 |
| extraction) | | | | |
| Nausea after | 0 | 0 | 1 | 3 |
| electrodiagnostic tests | | | | |
| Nausea during | 0 | 0 | 1 | 1 |
| microperimetry test | | | | |
| Tiredness | 0 | 0 | 4 | 6 |

Of the 40 participants, 38 (95%) completed sleep diaries during the dosing period, of whom 73% completed diaries for all three months. Of those participants, 26 reported discomfort and slippage as the primary issue concerning the mask. Only 3 participants who completed the sleep diaries reported the light as one of the issues, and only at month one; all 3 completed the trial.

Modifications and improvements can be incorporated herein without departing from the scope of the invention.

1. A method of light treatment therapy of an eye, the method comprising the step of administering light to the eye, the light having a wavelength of from 495 to 505 nm.

2. A method according to claim 1, the method being a treatment for one or more of a retinal disease, myopia, hyperopia, Age-Related Macular Degeneration, dry Age-Related Macular Degeneration, wet Age-Related Macular Degeneration, Diabetic Retinopathy, hypoxia, and hypoxia caused by dark adaptation.

3. A method according to claim 1, wherein the step of administering light to the eye is through closed eye lids.

4. A method according to claim 1, wherein the light is generated by one or more of an electroluminescent emitter, light emitting device (LED), light emitting cell (LEC), light emitting electrochemical cell (LEEC), and organic light emitting device (OLED).

5. A method according to claim 2, wherein a mask is used to administer light to the eye.

6. A method according to claim 1, wherein the wavelength of the light peaks at 504 nm or 498 nm.

7. A method according to claim 1, wherein the step of administering light to the eye lasts for from 4 to 8 hours.

8. A method according to claim 1, wherein the light has a frequency of from 526 to 606 THz.

9. A method according to claim 1, wherein the light has a photon energy of from 2.17 to 2.50 eV.

10. A method according to claim 1, wherein the light has a luminance of 50 cd/m² or 80 cd/m².

11. A method according to claim 1, wherein the light has a radiance from 0.06 mW/sr/m² to 0.2 mW/sr/m².

12. A method of improving eyesight in a healthy person, the method comprising the step of administering light to an eye, the light having a wavelength of from 495 to 505 nm.

13. A method according to claim 12, wherein the wavelength of the light peaks from 498 to 504 nm.

14. A method according to claim 12, wherein the wavelength of the light peaks at 504 nm or 498 nm.

15. A method according to claim 12, the method being a treatment for one or more of a retinal disease, myopia, Age-Related Macular Degeneration, dry Age-Related Macular Degeneration, wet Age-Related Macular Degeneration, Diabetic Retinopathy, hypoxia, and hypoxia caused by dark adaptation.

16. A method of preparing a healthy person for light treatment therapy of an eye, the method comprising the step of placing onto the person a mask having a light source for emitting light having a wavelength of from 495 to 505 nm.

17. A method according to claim 16, wherein the method further includes the step of selecting a healthy person who could benefit from the light treatment therapy.

18. A method according to claim 16, wherein the wavelength of the light peaks from 498 to 504 nm.

19. A method according to claim 16, wherein the wavelength of the light peaks at 504 nm or 498 nm.

20. A method according to claim 16, the method being a treatment for one or more of a retinal disease, myopia, hyperopia, Age-Related Macular Degeneration, dry Age-Related Macular Degeneration, wet Age-Related Macular Degeneration, Diabetic Retinopathy, hypoxia, and hypoxia caused by dark adaptation.
